(19) United States
(12) Patent Application Publication
(10) Pub. No.: US 2025/0125023 A1
(43) Pub. Date: Apr. 17, 2025

(54) COMPUTER IMPLEMENTED TECHNIQUES FOR SAMPLE OPTIMIZATION

(71) Applicant: Optum Services (Ireland) Limited, Dublin (IE)

(72) Inventors: Veronica AISPURO, Rancho Cucamonga, CA (US); Ciarán McKENNA, Dublin (IE); Guanghui CHEN, Allen, TX (US)

(21) Appl. No.: 18/486,270

(22) Filed: Oct. 13, 2023

(51) Int. Cl. G16H 10/40; G06F 17/18

(52) U.S. Cl. CPC G16H 10/40; G06F 17/18

(57) ABSTRACT

Systems and methods are disclosed for an automated process for determining an optimal sample size for a contract. The method includes receiving response data including response scores to queries from data collection objects. A sampling distribution is determined for each of sample sizes based on the response scores. A significance threshold and a reliability coefficient is determined for each of the sample sizes. A probability for each of star values for the sample sizes is determined based on a cumulative distributive function of the sampling distribution and parameters of an adjustment grid. An expected star value for each of the sample sizes is determined based on the probability determined for each of the star values. The expected star value is presented in a user interface of a device, wherein at least one of the sample sizes with a highest expected star value is recommended as the optimal sample size.

### TECHNICAL FIELD

This present disclosure relates generally to the field of data analysis. In particular, the disclosure relates to processing and analyzing patient experience data using statistical and logical methods for sample optimization.

### BACKGROUND

Healthcare service providers are continuously trying to make medical care more patient-centered. One of the approaches is to survey patients about their care experiences and utilize the survey results to guide improvement efforts. For example, Consumer Assessment of Healthcare Providers and Systems (CAHPS) surveys are used for obtaining patient assessments of healthcare facilities and providers. The Centers for Medicare & Medicaid Services (CMS) requires certain providers and facilities to participate in the CAHPS surveys to measure care experiences from the patient's perspective. CAHPS scores are also utilized in calculating star ratings that may help consumers compare the quality of Medicare health and drug plans being offered to make informed health care decisions.

Despite the importance of CAHPS surveys for calculating star ratings, there are several challenges in conducting these surveys that may directly impact the calculation of star ratings, such as the declining response rates (e.g., fewer participants completing the surveys result in lesser insights in understanding the drivers of patient satisfaction with the healthcare providers and facilities), reduced speed in delivering survey results (e.g., because of the delay in delivering the results, trending areas of concern may go unrecognized for an extended period of time before any action is taken), a lack of context (e.g., CAHPS questions are standardized without the flexibility to incorporate workflows or additional questions based on survey responses, this precludes important contextual information needed to drive improvement), higher administration costs (e.g., traditional methods place an excessive financial and administrative burden on the providers), and the static nature of the CAHPS surveys making them suboptimal and incompatible with modern techniques. Hence, service providers are technically challenged in developing methods to ensure credibility, accuracy, and/or usefulness of healthcare data and CAHPS surveys for calculating star ratings.

### SUMMARY OF THE DISCLOSURE

The present disclosure solves this problem and/or other problems described above or elsewhere in the present disclosure and improves the state of conventional data analysis techniques for healthcare data.

Current approaches for conducting CAHPS surveys and calculating sample size recommendations are based on static methods that are suboptimal, and their level of modeling and calculations is basic. For example, current approaches utilize a non-probabilistic method that generates a fixed line graph, a suboptimal solution that does not account for the fact that the sample is from a bigger population. These approaches do not take into account statistical error from sampling, thereby yield poor results. Furthermore, the output of the current approaches lacks accuracy because of sampling variability. In addition, these approaches lack efficiency because the number of iterations required to match the performance of the proposed methodology is significant.

In some embodiments, a system for an automated process for determining an optimal sample size for a contract is disclosed. The system includes: one or more processors; and at least one non-transitory computer readable medium storing instructions which, when executed by the one or more processors, cause the one or more processors to perform operations including: receiving response data from data collection objects, wherein the response data includes response scores to queries in the data collection objects; determining a sampling distribution for each of one or more sample sizes based on the response scores; determining a significance threshold and a reliability coefficient for each of the one or more sample sizes; determining a probability for each of one or more star values for the one or more sample sizes based on a cumulative distributive function (CDF) of the sampling distribution and one or more parameters of an adjustment grid; determining an expected star value for each of the one or more sample sizes based on the probability determined for each of the one or more star values; and causing a presentation of the expected star value determined for each of the one or more sample sizes in a user interface of a device, wherein at least one of the one or more sample sizes with a highest expected star value is recommended as the optimal sample size.

In some embodiments, a computer-implemented method for an automated process for determining an optimal sample size for a contract is disclosed. The computer-implemented method includes: receiving, by one or more processors, response data from data collection objects, wherein the response data includes response scores to queries in the data collection objects; determining, by the one or more processors, a sampling distribution for each of one or more sample sizes based on the response scores; determining, by the one or more processors, a significance threshold and a reliability coefficient for each of the one or more sample sizes; determining, by the one or more processors, a probability for each of one or more star values for the one or more sample sizes based on a CDF of the sampling distribution and one or more parameters of an adjustment grid; determining, by the one or more processors, an expected star value for each of the one or more sample sizes based on the probability determined for each of the one or more star values; and causing, by the one or more processors, a presentation of the expected star value calculated for each of the one or more sample sizes in a user interface of a device, wherein at least one of the one or more sample sizes with a highest expected star value is recommended as the optimal sample size.

In some embodiments, a non-transitory computer readable medium for an automated process for determining an optimal sample size for a contract is disclosed. The non-transitory computer readable medium stores instructions which, when executed by one or more processors, cause the one or more processors to perform operations including: receiving response data from data collection objects, wherein the response data includes response scores to queries in the data collection objects; determining a sampling distribution for each of one or more sample sizes based on the response scores; determining a significance threshold and a reliability coefficient for each of the one or more sample sizes; determining a probability for each of one or more star values for the one or more sample sizes based on a CDF of the sampling distribution and one or more parameters of an adjustment grid; determining an expected star value for each of the one or more sample sizes based on the probability determined for each of the one or more star values; and causing a presentation of the expected star value determined for each of the one or more sample sizes in a user interface of a device, wherein at least one of the one or more sample sizes with a highest expected star value is recommended as the optimal sample size.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of an automated system for selecting an optimal sample size that maximizes final star ratings for a given contract by minimizing the number of star downward adjustments and maximizing star upward adjustments, according to aspects of the disclosure.

FIG. 2 is a diagram of the components of an optimization platform, according to one example embodiment.

FIG. 3A is a flowchart of a process for an automated process to select an optimal sample size that maximizes final star ratings for a given contract by minimizing the number of star downward adjustments and maximizing star upward adjustments, according to aspects of the disclosure.

FIGS. 3B-3D illustrate a star value adjustment process, according to aspects of the disclosure.

FIG. 4A is a graph that illustrates the impact of sample size on significance and reliability metrics, according to aspects of the disclosure.

FIG. 4B is a graph that illustrates the impact of sample size on cutpoint star volatility, according to aspects of the disclosure.

FIG. 5 is a graph that illustrates sampling distribution for a contract-measure for determining an expected star value, according to aspects of the disclosure.

FIG. 6A is a user interface diagram that illustrates an optimal sample size, according to aspects of the disclosure.

FIG. 6B is a user interface diagram that illustrates an expected contract star by sample size and mean adjustment using a parametric method, according to aspects of the disclosure.

FIG. 7 illustrates an implementation of a computer system that executes techniques presented herein.

### DETAILED DESCRIPTION OF EMBODIMENTS

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for an automated process to select an optimal sample size that maximizes final star ratings for a given contract by minimizing the number of star downward adjustments and maximizing star upward adjustments.

Healthcare providers are continuously trying to improve their clinical, operational, and financial performance. A critical tool in doing so is the ability to efficiently analyze volumes of comprehensive data for each of the foregoing aspects. The CMS develops, implements, and administers several different patient experience surveys that ask patients about their experiences with their healthcare providers and plans. Many of the CMS patient experience surveys are within the CAHPS family of surveys. The CAHPS surveys follow scientific principles in survey design and development to reliably assess the experiences of a large sample of patients. The CAHPS surveys use standardized questions and data collection protocols to ensure that information can be compared across healthcare settings.

The CMS stars rating system includes a contract-level rating system, wherein five stars represent excellent performance, four stars represent above-average performance, three stars represent average performance, two stars represent below-average performance, and one star represents poor performance. Individual CMS measures are selected from a range of different rating systems, including, but not limited to, CAHPS. Given the importance of achieving a higher star rating, it is critical for the providers to be able to understand CAHPS at a member level. However, given the anonymized nature of the CMS survey process, CAHPS measures are challenging to improve.

The retrospective data captured by the CAHPS surveys has its limits. For example, CAHPS regulations stipulate that patients must be surveyed between 48 hours and six weeks after discharge. The time it takes for their responses to be recorded and reported to the providers, the data becomes stale. These conventional methods do not provide real-time or near real-time predicted assessments on an ongoing basis that provide service providers with trending information associated with the services provided.

As discussed, the CAHPS surveys assess a participant's experience with their health plan, health care, and drug plan. Conventionally, these surveys are conducted during a specific time period (e.g., March to June) with two mail survey attempts and up to 5 telephone attempts via interactive voice response (IVR) technology (e.g., a call by a contact center agent). The eligible participants for the CAHPS survey must be with the health plan for at least six months before the survey period. In one instance, the CAHPS survey includes 9 experience measures, there are approximately seventy questions, and twenty-one of these questions are directly utilized for CAHPS experience measures considered for the CMS star ratings. They include member rating of their drug/health plan and healthcare, ease of use of prescription drug plan, customer service, getting care quickly, getting needed care, and care coordination. In one instance, healthcare providers (e.g., healthcare payers) are allowed to select the sample size of membership surveyed for each contract with a minimum sample size of 800 and a maximum sample size of 5,000. Since sample size influences significance, reliability, standard error, and cutpoint star volatility, an automated process for determining an optimal sample size to optimize CAHPS survey stars is needed. There is a need for a method that selects a sample size that maximizes the CAHPS final star rating for a given contract by minimizing the number of star downward adjustments and maximizing star upward adjustments.

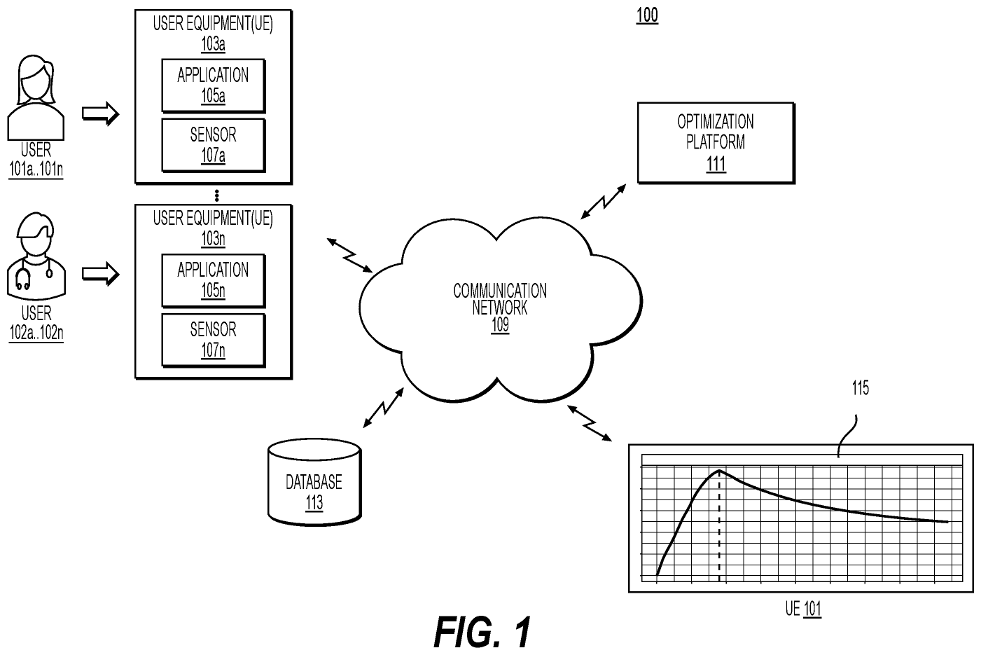

To address these technical challenges, FIG. 1 implements modern data processing and analyzing capabilities into methods and systems for an automated process to select an optimal sample size that maximizes final star ratings for a given contract by minimizing the number of star downward adjustments and maximizing star upward adjustments. FIG. 1, an example architecture of one or more example embodiments of the present disclosure, includes a system 100 that comprises user 101a-101n (collectively referred to as user 101), user 102a-102n (collectively referred to as user 102), user equipment (UE) 103a-103n (collectively referred to as UE 103) that includes applications 105a-105n (collectively referred to as an application 105) and sensors 107a-107n (collectively referred to as a sensor 107), a communication network 109, the optimization platform 111, and a database 113. In one instance, the system 100 generates a simulation in the UE 103 associated with the user 102 to demonstrate the optimal contract sample size as a function of the mean star rating for contracts across the CAHPS measures. The system 100 by determining the optimal contract sample size reduces oversampling, which can result in the saving of resources. The system 100 also generates a graph in the UE 103 associated with the user 102 to demonstrate the impact of sample size on cut-point star volatility. In another instance, the system 100 provides an automated process for selecting an optimal sample size (e.g., in real-time or near real-time), thereby overcoming the identified difficulties of declining response rates and the reduced speed in delivering the survey results. In a further instance, the system 100 provides contextual information determination services to extract relevant contextual information needed to drive improvement from the database 113.

In one embodiment, the user 101 is a person or a group of people interacting with a user interface or a web interface of the UE 103 to access healthcare-related surveys (e.g., CAHPS surveys) regarding their experience with healthcare providers and plans. For example, the user 101 includes registered patients that satisfy CAHPS requirements, e.g., patients with a health plan for at least six months before the survey period, patients who have visited the participating healthcare providers during specified time intervals, etc. In one example embodiment, survey questions are presented to the user 101 by textual, graphic, auditory, or any other means in the user interface or a web interface of the UE 103. The user 101 can provide response data to healthcare-related surveys directly via the UE 103. Alternatively, the healthcare-related surveys may be administered by mail or telephone. Any other modes for conducting surveys may be implemented per requirement.

In one embodiment, the user 102 are administrators, analysts, contract managers, data specialists, survey team, and other individuals or groups assigned with the responsibility for CMS measures. In one embodiment, the optimization platform 111 provides the user 102 with recommendations on optimal sample size for a contract. In one embodiment, the optimization platform 111 guides the user 102 with respect to goals and initiatives that are achievable based on CMS measures and predicted CMS stars rating. For example, the optimization platform 111 presents interface 115, which provides data on corresponding CMS star ratings in the UE 103 associated with the user 102.

In one embodiment, the UE 103 includes but is not restricted to, any type of mobile terminal, wireless terminal, fixed terminal, or portable terminal. Examples of the UE 103 include hand-held computers, desktop computers, laptop computers, wireless communication devices, cell phones, smartphones, mobile communications devices, a Personal Communication System (PCS) device, tablets, server computers, gateway computers, or any combination thereof, including the accessories and peripherals of these devices, or any combination thereof. In addition, the UE 103 facilitates various input means for receiving and generating information, including, but not restricted to, a touch screen capability, a keyboard, and keypad data entry, a voice-based input mechanism, and the like. Any known and future implementations of the UE 103 are also applicable. In one instance, the UE 103 generates various presentations for the user 101 to take online healthcare-related surveys and provides assistance for making this process as effective as possible. In one example embodiment, healthcare-related survey forms are electronic forms with a plurality of data fields. The user 101 input, via a user interface of the UE 103, ratings and comments into the plurality of data fields regarding their experience with healthcare providers. Examples of electronic forms include portable document format, word document, rich text format (RTF), fillable web form (e.g., a webpage with fillable data fields), or any other suitable electronic format as would be understood in the art. There are multiple ways of entering information into the healthcare-related survey forms, including typing text in the word document, filling in a data field of a form in portable document format, filling in a data field of a web form, etc. Alternatively, the user 101 receives the survey forms via mail or prints the electronic forms, enters the information by hand, and submit a scanned copy, via the UE 103, for further processing.

In one embodiment, the application 105 includes various applications such as, but not restricted to, content provisioning applications, software applications, networking applications, multimedia applications, media player applications, storage services, contextual information determination services, notification services, and the like. In one embodiment, one of the application 105 at the UE 103 acts as a client for the optimization platform 111 and performs one or more functions associated with the functions of the optimization platform 111 by interacting with the optimization platform 111 over the communication network 109.

By way of example, each sensor 107 includes any type of sensor. In one embodiment, the sensors 107 include, for example, a network detection sensor for detecting wireless signals or receivers for different short-range communications (e.g., Bluetooth, Wi-Fi, Li-Fi, near field communication (NFC), etc.) from the communication network 109, a camera/imaging sensor for gathering image data (e.g., images of completed surveys, video recordings of completed surveys, etc.), an audio recorder for gathering audio data (e.g., audio recordings of telephonic interviews), and the like.

In one embodiment, various elements of the system 100 communicate with each other through the communication network 109. The communication network 109 supports a variety of different communication protocols and communication techniques. In one embodiment, the communication network 109 allows the UE 103 to communicate with the optimization platform 111. The communication network 109 of the system 100 includes one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

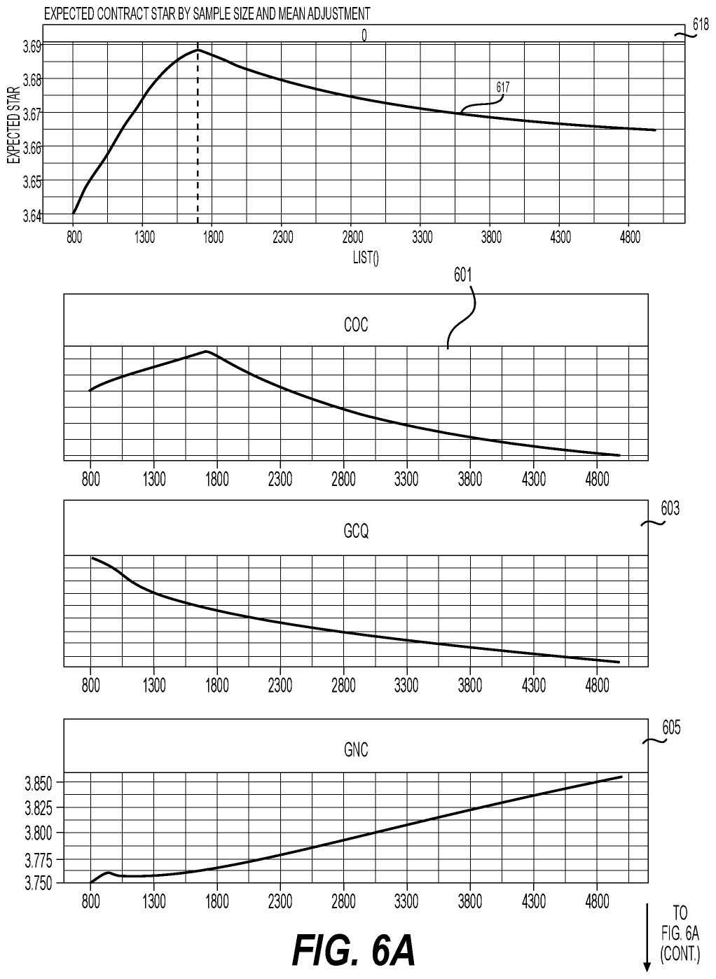

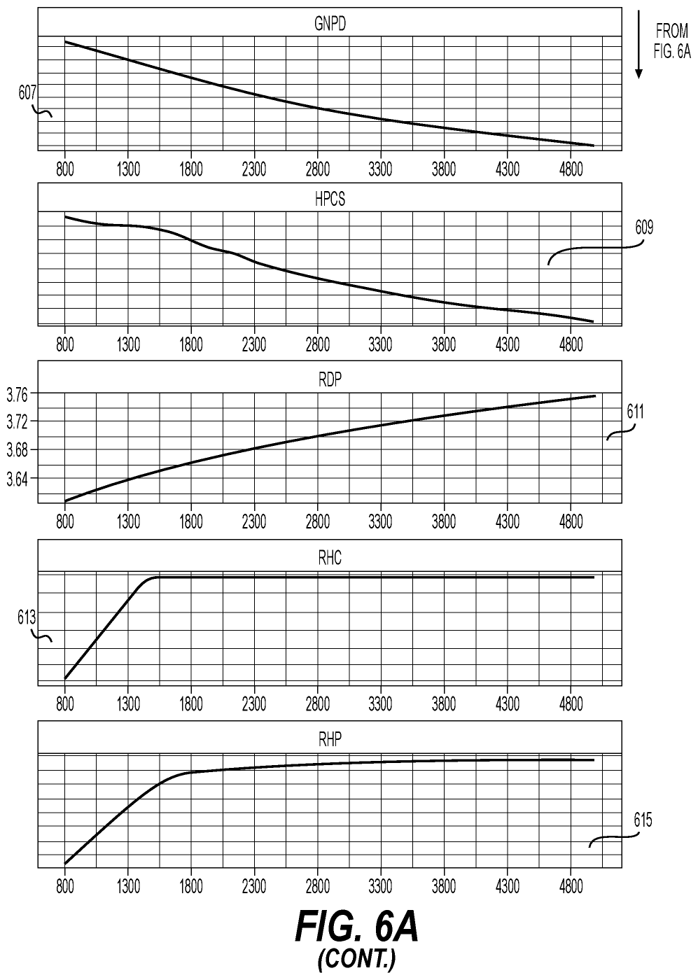

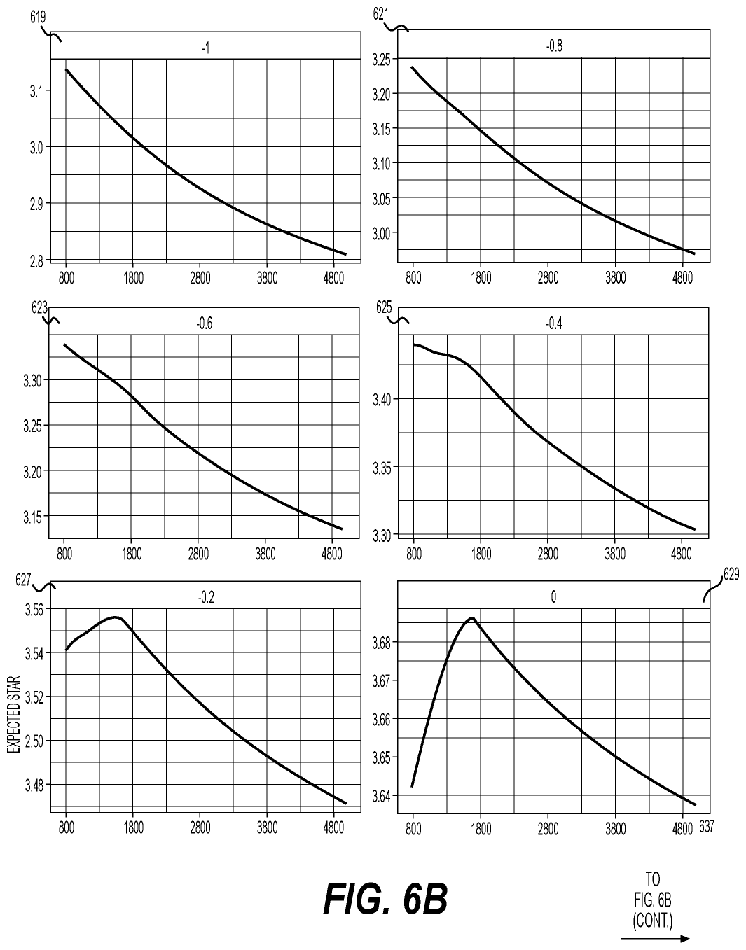

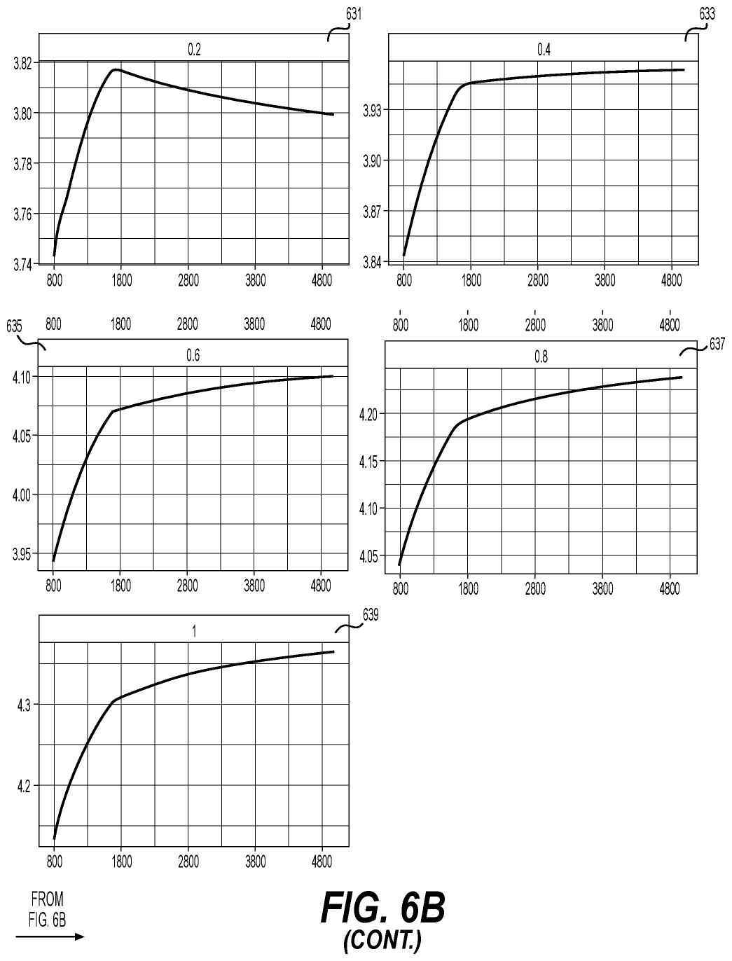

In one embodiment, the optimization platform 111 is a platform with multiple interconnected components. The optimization platform 111 includes one or more servers, intelligent networking devices, computing devices, components, and corresponding software for an automated process to select an optimal sample size that maximizes final star ratings for a given contract by minimizing the number of star downward adjustments and maximizing star upward adjustments. In one embodiment, the optimization platform 111 calculates sample size to optimize CAHPS survey stars. The optimization platform 111 utilizes a probabilistic approach and/or parametric processes to account for sampling variability impacts on star rating adjustments. Such implementation of the probabilistic approach and/or parametric processes generates informed graphs (as illustrated in FIGS. 6A and 6B).

In one instance, CMS has a minimum sample size of 800. If a service provider samples 800 participants, they may not receive responses from all the participants. Generally, there is a tendency for the service providers to oversample (e.g., increasing the sample size) to increase confidence (e.g., the mean better represents the true mean) and to meet minimal sample requirements to have a reportable sample. In one instance, by determining the optimal sample size, the optimization platform 111 reduces oversampling, which can result in the saving of resources. In another instance, the optimization platform 111 determines oversampling to be unnecessary and provides a recommendation. For example, the optimization platform 111 determines if the service provider oversamples, they may receive a two-star value. Hence, it is better to have no score added, than having a low score added that brings the average down. Alternatively, the optimization platform 111 determines a need for oversampling based on the response rate. A detailed description of the optimization platform 111 is provided in the descriptions below.

In one embodiment, the database 113 is any type of database, such as relational, hierarchical, object-oriented, and/or the like, wherein data are organized in any suitable manner, including data tables or lookup tables. In one embodiment, the database 113 accesses or stores content associated with the user 101, the UE 103, and the optimization platform 111, and manages multiple types of information that provide means for aiding in the content provisioning and sharing process. It is understood that any other suitable data may be included in the database 113. In one instance, the database 113 stores completed healthcare-related surveys, CMS star data including historical CMS measures and star ratings, contextual information associated with the user 101, etc. The historical CMS measures and star ratings are typically associated with contracts that have already been subject to the CMS star rating process. Accordingly, the database 113 facilitates comparisons of CAHPS survey results. In another embodiment, the database 113 includes a machine-learning based training database with a pre-defined mapping defining a relationship between various input parameters and output parameters based on various statistical methods. In one embodiment, the training database includes a dataset that includes data collections that are not subject-specific, e.g., data collections based on population-wide observations, local, regional, or super-regional observations, and the like. In an embodiment, the training database is routinely updated and/or supplemented based on machine learning methods.

By way of example, the UE 103 and the optimization platform 111 communicate with each other and other components of the communication network 109 using well known, new or still developing protocols. In this context, a protocol includes a set of rules defining how the network nodes within the communication network 109 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers as defined by the OSI Reference Model.

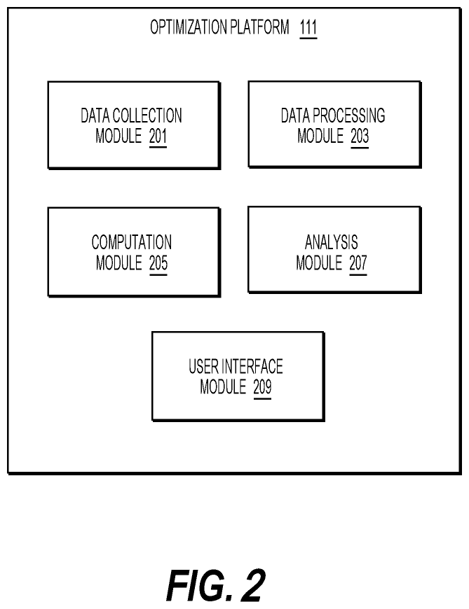

FIG. 2 is a diagram of the components of the optimization platform 111, according to one example embodiment. As used herein, terms such as “component” or “module” generally encompass hardware and/or software, e.g., that a processor or the like may use to implement associated functionality. By way of example, the optimization platform 111 includes one or more components for an automated process to select an optimal sample size that maximizes final star ratings for a given contract by minimizing the number of star downward adjustments and maximizing star upward adjustments. It is contemplated that the functions of these components may be combined in one or more components or performed by other components of equivalent functionality. In one embodiment, the optimization platform 111 comprises a data collection module 201, a data processing module 203, a computation module 205, an analysis module 207, a user interface module 209, or any combination thereof.

In one embodiment, the data collection module 201 collects relevant data (e.g., CAHPS survey data from the UE 103, historical CAHPS survey data stored in the database 113, historical CMS star ratings stored in the database 113, etc.) through various data collection techniques. In one embodiment, the data collection module 201 uses a web-crawling and/or other types of data collection mechanism to access various databases or other information sources (e.g., government websites providing electronic forms for users to take CAHPS surveys) to collect the relevant data. In one embodiment, the data collection module 201 includes various software applications, e.g., data mining applications in Extended Meta Language (XML) that automatically search for and return relevant data regarding the CAHPS survey and/or CMS ratings.

In one embodiment, the data processing module 203 processes the data collected by the data collection module 201. The data processing module 203 parses and arranges the data into a common format that can be easily processed by other modules and platforms. For example, the data processing techniques include, but are not limited to, an optical character recognition (OCR) technique, an NLP technique, or a data cleansing technique. In one instance, the OCR technique is applied to the scanned survey forms, pictures of the scanned survey forms that include text, and/or the like, to generate electronic data, e.g., text data. The data processing module 203 converts the printed texts to electronic data for editing, searching, compactly storing, displaying online, and/or using in machine processes such as cognitive computing, machine translation, (extracted) text-to-speech, key data and text mining, and/or the like. In one instance, the NLP technique is applied to analyze, understand, and derive meaning from the texts written by the user 101 in the scanned survey forms. In one instance, the data cleansing technique detects and corrects inconsistencies originally caused by user entry errors or corruption during transmission or storage. For example, the data cleansing technique detects corrupt or inaccurate data, and then replaces, modifies, or deletes the corrupt or inaccurate data. The data cleansing technique also includes cleaning the data by cross-checking the data with a validated data set, and standardizing the data by changing a reference data set to a new standard.

In one embodiment, the computation module 205 receives the processed data from the data processing module 203, and is configured to perform calculations on the processed data utilizing various equations for data analysis and interpretation. In one instance, each CAHPS survey produces several measures of the patient experience. These measures include composite measures, which combine two or more related survey items; rating measures, which reflect respondents' ratings on a scale of 0 to 10; and single-item measures. Each measure receives a score based on responses to questions (or sub-questions). For example, a Rating of Health Plan (RHP) measure asks the user 101 to rate their health plan in integer values (e.g., from 01-10). The user 101 chooses a score of 8, and their score is given by (8/10)*100=80. However, not all survey questions are asked on 1-10 increments, hence the computation module 205 adjusts the values measured on different scales to a notionally common scale (e.g., normalizes the scores to a 0-100 scale).

In one instance, CAHPS survey responses are aggregated at the contract-measure level to a score in the [0,100] scale. These scores are then converted into measure stars and contribute to a contract's overall rating. In one instance, consider a CAHPS contract-measure with mean μ, standard deviation σ, sample size n and response rate q. The respondent size for the contract is given by r=qn.

In accordance with the Central Limit Theorem, the sampling distribution S for the contract with new sample size n_{new}, is approximated by:

$$S ∼ N ( μ , \frac{{σ}^{2}}{{r}_{n e w}} ) \qquad equation 1$$

In one embodiment, the computation module 205 establishes an appropriate mean sampling distribution for a given sample size based on equation 1.

In one instance, the standard error for the contract based on the new sample size is given by:

$$S E = \frac{σ}{\sqrt{{r}_{n e w}}} \qquad equation 2$$

In one instance, the re-calculated reliability coefficient is given by:

$$R = \frac{I}{I + {SE}^{2}} \qquad equation 3$$

where I is the variance between contracts across industry.

In one instance, the thresholds for contract significance levels based on the new sample size are given by:

$$s i {g}_{a b ove / below} = \pm 1.96 S {E}_{D N} + M \qquad equation 4$$

where M is the industry mean across contracts and

$$S {E}_{D N} = \sqrt{{( \frac{σ ( P - 1 )}{P} )}^{2} ( \frac{1}{{r}_{n e w}} - \frac{1}{r} ) + {( \frac{σ}{r} )}^{2}} \qquad equation 5$$

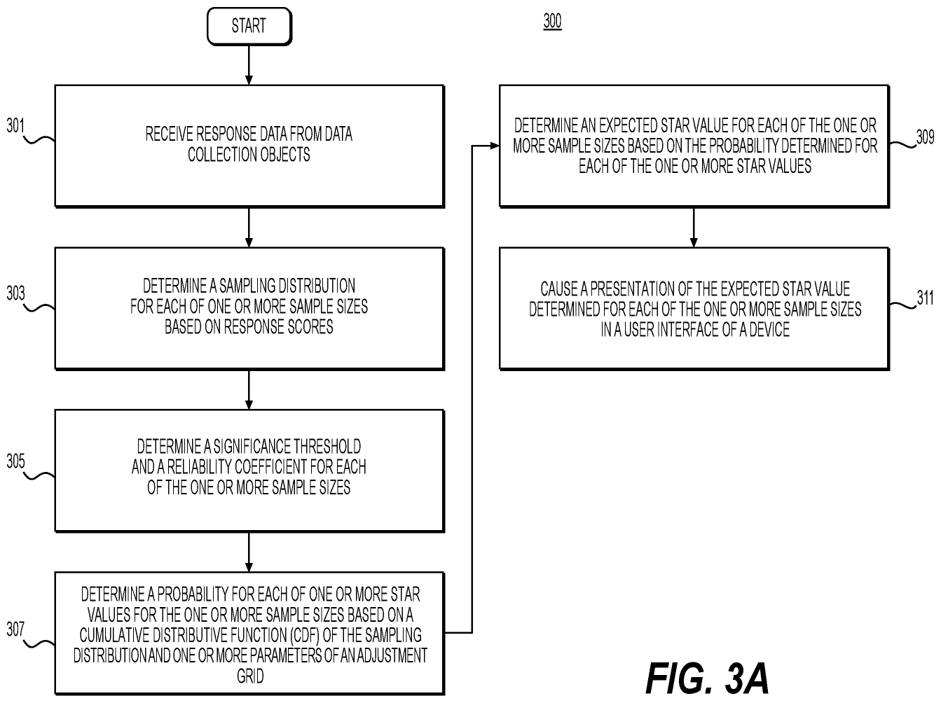

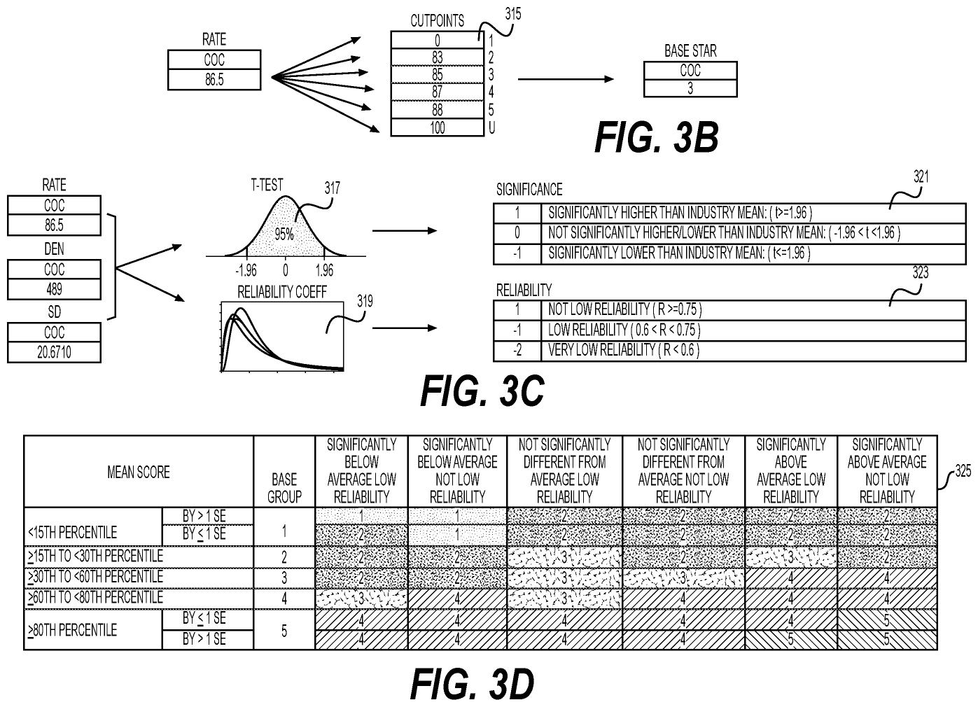

is the recalculated standard error of the difference between M and μ (based on the new sample size n_{new}). In one embodiment, the computation module 205 recalculates the significance and reliability thresholds based on equations 2, 3, 4, and 5 and statistical tests (e.g., t-test and reliability coefficient). In one embodiment, the computation module 205 uses the cumulative distribution function (CDF) of S to calculate the probability of obtaining a given star based on the parameters of the CMS adjustment grid (as illustrated in FIG. 3D).

In one embodiment, the computation module 205 calculates for each measure and allowed sample size, the area under the curve of the probability density function (PDF) of S, partitioned by the measure cutpoints, sig_{above/below} thresholds and new reliability assignment according to the CAHPS adjustment grid to obtain the probability of the contract hitting each star rating. In one instance, calculating the area under the curve of the PDF of S, partitioned by the measure cutpoints, sig_{above/below} thresholds, and new reliability assignment according to the CAHPS adjustment grid to obtain the probability of the contract hitting each star rating may be comparable to utilizing the CDF of S to calculate the probability of obtaining a given star based on the parameters of the CMS adjustment grid.

In one embodiment, the computation module 205 calculates the expected star value for the measure by taking the sum of the product of the probability and the star value itself:

$$E = {Σ}_{s = 1}^{5} P ( s ) * s \qquad equation 6$$

where P(s) is the probability of obtaining a star=s.

In one embodiment, the computation module 205 calculates the mean across each expected measure star to obtain the expected contract star.

In one embodiment, the analysis module 207 is configured to process data from the computation module 205 to make an informed decision. The analysis module 207 includes code for processing data and displaying visualized information in a user interface as a graph. For example, the analysis module 207 obtains a set of summary statistics from the computation module 205 to output charts displaying the expected star value as a function of the selected sample size at both the contract level and/or measure level. In one embodiment, the analysis module 207 is implemented to provide logic, control, coordination, command, signaling, and other functions and features. For example, although the other modules may perform their actual tasks, the analysis module 207 determines when and how those tasks are performed or otherwise direct the other modules to perform the task. In one instance, the analysis module 207 instructs the data collection module 201 to collect specific data or the data processing module 203 to process specific data In one instance, the analysis module 207 instructs the user interface module to present graphs displaying the expected star value.

In one embodiment, the user interface module 209 enables a presentation of a graphical user interface (GUI) in the UE 103. The user interface module 209 employs various application programming interfaces (APIs) or other function calls corresponding to the application 105 on the UE 103, thus enabling the display of graphics primitives such as icons, menus, buttons, data entry fields, graphs, charts, tables, etc. In one instance, the user interface module 209 causes interfacing of guidance information with the user 101 to include, at least in part, one or more annotations, audio messages, video messages, or a combination thereof while completing healthcare-related surveys. In another instance, the user interface module 209 causes interfacing of guidance information with the user 102 to include, at least in part, one or more annotations, audio messages, video messages, or a combination thereof while presenting the interface 115 which provides data on CAHPS surveys and/or CMS star ratings. In one embodiment, the user interface module 209 comprises a variety of interfaces, for example, interfaces for data input and output devices, referred to as I/O devices, storage devices, and the like. Still further, the user interface module 209 is configured to operate in connection with augmented reality (AR) processing techniques, wherein various applications, graphic elements, and features interact.

The above presented modules and components of the optimization platform 111 may be implemented in hardware, firmware, software, or a combination thereof. Though depicted as a separate entity in FIG. 2, it is contemplated that the optimization platform 111 may be implemented for direct operation by respective UE 103. As such, the optimization platform 111 may generate direct signal inputs by way of the operating system of the UE 103. In another embodiment, one or more of the modules 201-209 may be implemented for operation by respective UEs, as the optimization platform 111, or a combination thereof. The various executions presented herein contemplate any and all arrangements and models.

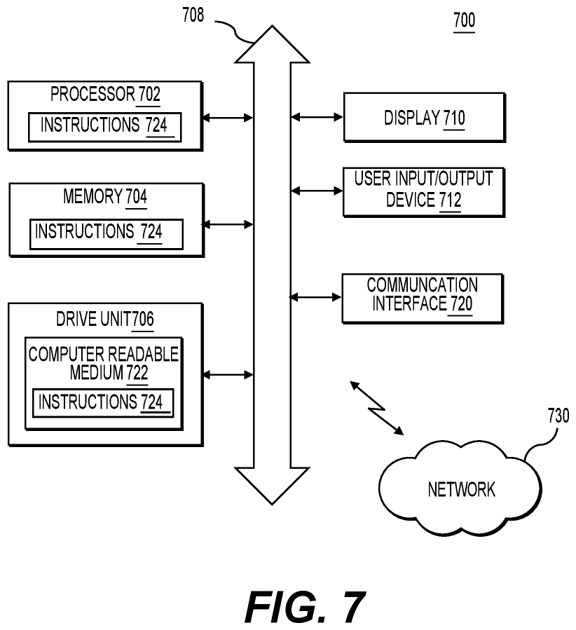

FIG. 3A is a flowchart of a process for an automated process to select an optimal sample size that maximizes final star ratings for a given contract by minimizing the number of star downward adjustments and maximizing star upward adjustments, according to aspects of the disclosure. In various embodiments, the optimization platform 111 and/or any of the modules 201-209 performs one or more portions of the process 300 and are implemented using, for instance, a chip set including a processor and a memory as shown in FIG. 7. As such, the optimization platform 111 and/or any of modules 201-209 provide means for accomplishing various parts of the process 300, as well as means for accomplishing embodiments of other processes described herein in conjunction with other components of the system 100. Although the process 300 is illustrated and described as a sequence of steps, it is contemplated that various embodiments of the process 300 are performed in any order or combination and need not include all of the illustrated steps.

In step 301, the optimization platform 111, via one or more processors 702, receives response data from data collection objects. In one embodiment, the response data includes response scores to queries in the data collection objects. In one embodiment, the data collection objects include CAHPS. In one instance, each sample is at a contract level. Each contract has a set of members (e.g., 100,000, 1,000,000, and so on) and a random group of members is selected (e.g., exclude members who are not eligible, such as members who are not continuously enrolled with the health plan for 6 months, etc.). For example, out of the 100,000 members, around 80,000 members are selected for CAHPS. The bias is who is going to respond, and finding the members. In one instance, the user 101 participating in CAHPS receives queries (e.g., in the last 6 months, how often did your health plan's customer service give you the information or help you needed? using any number from 0 to 10, where 0 is the worst health care possible and 10 is the best health care possible, what number would you use to rate all your health care in the last 6 months? etc.). The response from the user 101 to the queries is scored, via the usual methodology, to generate response scores. In one instance, the responses are aggregated and normalized at the contract-measure level to a score in the [0,100] scale. Alternatively, the response score may be compared to cutpoints to generate a base star value that is further adjusted based on additional tests/metrics (e.g., significance, reliability, and contract standard error). As illustrated in FIG. 3B, the response score of 86.5 is compared to cutpoints 315 to determine a base star value of 3. Wherever the response score lies between the values of cutpoints 315, a base star value is provided (e.g., an integer value between 1 to 5). These scores are then converted into measure star values that contribute to a contract's overall rating.

In step 303, the optimization platform 111, via the one or more processors 702 utilizing the computation module 205, determines a sampling distribution for each of one or more sample sizes based on the response scores. As discussed, the sampling distribution S for the contract with new sample size n_{new}, is approximated by:

$$S ∼ N ( μ , \frac{{σ}^{2}}{{r}_{n e w}} )$$

In step 305, the optimization platform 111, via the processor 702 utilizing the computation module 205, determines (e.g., calculates) a significance threshold(s) and a reliability coefficient for each of the one or more sample sizes. As discussed, the significance and reliability thresholds are recalculated utilizing statistical tests and formulas, for example equations 4 and 5:

$${sig}_{↑ / ↓} = \pm 1.96 S {E}_{D N} + M where {SE}_{D N} = \sqrt{{( \frac{σ ( P - 1 )}{P} )}^{2} ( \frac{1}{{n}_{n e w}} - \frac{1}{n} ) + {( \frac{σ}{n} )}^{2}}$$

In one embodiment, the significance threshold(s) are an output of a two sided t-test that compares the mean of the contract to an average of one or more other contracts (e.g., the national average). In one embodiment, the reliability coefficient compares a variance of the contract to variances between one or more other contracts (e.g., variance between contracts across the industry), thereby measuring how distinguishable the contract score is from the industry scores. As illustrated in FIG. 3C, the response score of 86.5, the denominator or respondent size of 489, and/or standard deviation of 20.6710 is inputted, via the usual methodology, to two sided t-test 317 and/or reliability coefficient metric 319 to generate a significance threshold 321 and/or a reliability coefficient 323. In one instance, the base star, significance threshold, and/or reliability coefficient are compared to the adjustment grid 325 to adjust the base star. For example, the base star value is 3, the two sided t-test 317 outputs significantly below the industry mean (e.g., −1), and the reliability coefficient 323 outputs low reliability. Then, the base star value of 3 is adjusted down to 2 per the adjustment grid 325. In one instance, comparing star values to the adjustment grid 325 is an iterative process that occurs at various stages (e.g., steps of the process 300) while determining the optimal sample size for a contract.

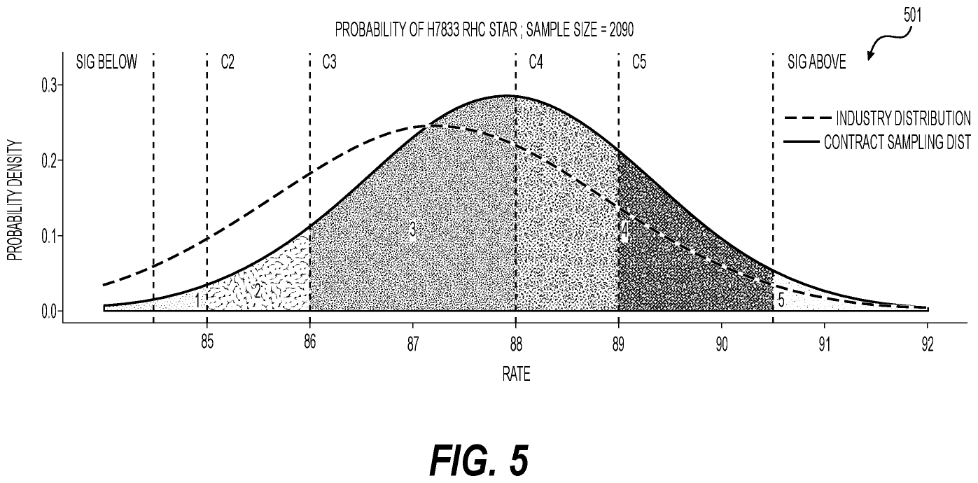

In step 307, the optimization platform 111, via the one or more processors 702 utilizing the computation module 205, determines (e.g., calculates) a probability for each of one or more star values for the one or more sample sizes based on a cumulative distributive function (CDF) of the sampling distribution and one or more parameters of the adjustment grid 325. In one embodiment, the optimization platform 111, via the one or more processors 702, determines a curve indicating the sampling distribution for the one or more sample sizes (e.g., the curve represented in chart 501 of FIG. 5, discussed in detail below). The optimization platform 111, via the one or more processors 702, determines a probability of the contract achieving each star value by determining (e.g., calculating) an area under the curve of a probability density function of the sampling distribution (e.g., areas 1, 2, 3, 4, and 5 in chart 501 of FIG. 5, discussed in detail below). In one instance, the curve is partitioned by at least one of a cutpoints (e.g., c2, c3, c4, and c5 in chart 501 of FIG. 5), a significance threshold, or a reliability assignment according to the adjustment grid 325. In one embodiment, the optimization platform 111, via the one or more processors 702, determines (e.g., calculates) a standard error for each of the one or more sample sizes, wherein the standard error measures variation in contract values, and wherein the standard error is based on the response scores and a user size (e.g., the respondent's size). In one embodiment, each contract value is a mean of the response scores to the queries in the data collection objects. In one embodiment, the user size is a product of a response rate and a sample size.

In step 309, the optimization platform 111, via the one or more processors 702, determines (e.g., calculates) an expected star value for each of the one or more sample sizes based on the probability determined for each of the one or more star values. In one embodiment, the optimization platform 111, via the one or more processors 702, determines (e.g., calculates) a mean across each expected measure star, wherein the expected measure star is a product of the probability for each star value and the star value. As discussed, contract expected star E=Σₛ₌₁⁵ P(s)*s where P(s) is the probability of obtaining a star=s.

In step 311, the optimization platform 111, via the one or more processors 702 utilizing the user interface module 209, causes a presentation of the expected star value determined for each of the one or more sample sizes in a user interface of the UE 103. In one embodiment, at least one of the one or more sample sizes with a highest expected star value is recommended as the optimal sample size. In one embodiment, the user interface includes a visual representation of the expected star value determined for each of the one or more sample sizes, as a function of the one or more sample sizes at a contract level and a measure level. In one instance, the optimization platform 111 displays, in real-time, the expected star value for each of the sample sizes, thereby resolving the issues of declining response rates and reduced speed in delivering survey results. In one instance, the optimization platform 111 utilizes a probabilistic method to generate flexible graphs that are updated, in real-time, to provide adequate context to incorporate workflows or additional questions based on survey responses. For example, this output is the mechanism by which healthcare providers (e.g., healthcare payers) may select sample size that is submitted to CMS for each contract for the CAHPS survey. The output is reviewed by user 102 (e.g., survey team) in conjunction with contract owners to choose a sample size that maximizes the projected CAHPS stars.

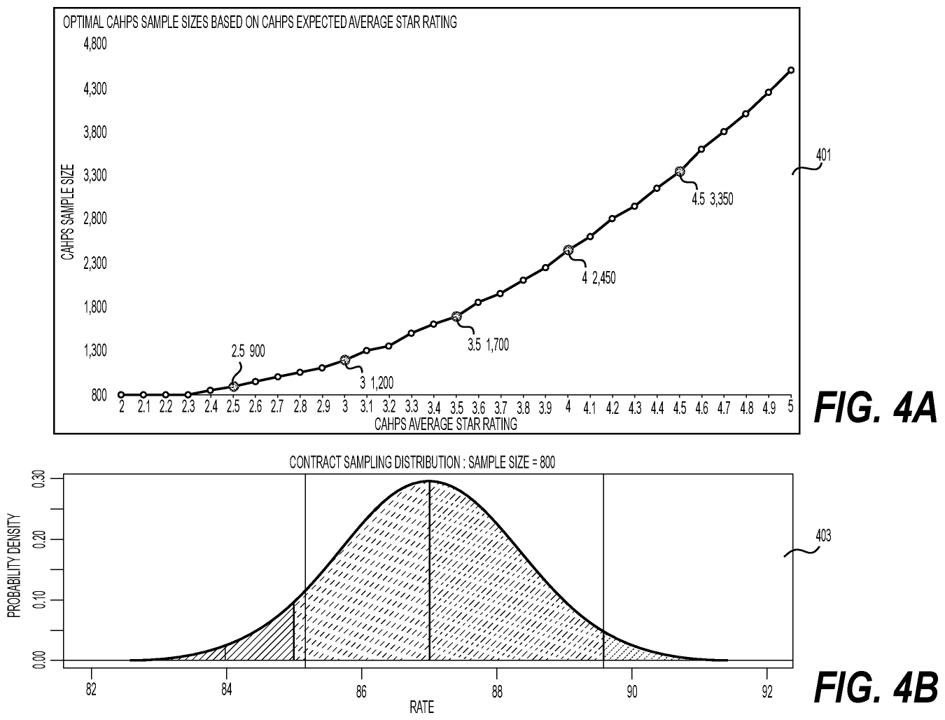

FIG. 4A is a graph that illustrates the impact of sample size on significance and reliability metrics, according to aspects of the disclosure. As discussed, the significance metric is based on a t-test, and larger sample sizes result in the outcome being more sensitive to differences between the contract and industry mean (due to shrinking standard error). Therefore, high performing contract-measures benefit from increased sensitivity since they are more likely to achieve a ‘significantly above’ assignment and not receive a downward star adjustment. The inverse is also true for low performing contract-measures as they are more likely to receive a “significantly below” assignment and receive a star deduction. In one embodiment, the optimization platform 111, utilizing the user interface module 209, generates a simulation 401 (e.g., a multiple probability simulation, a web-based simulation, or any other suitable simulation) in the UE 103 associated with the user 102 to demonstrate the optimal contract sample size as a function of the mean star rating for contracts across the CAHPS measures. In this example embodiment, an optimal contract sample size of 900 is recommended for CAHPS average star rating of 2.5, an optimal contract sample size of 1200 is recommended for CAHPS average star rating of 3, an optimal contract sample size of 1700 is recommended for CAHPS average star rating of 3.5, an optimal contract sample size of 2450 is recommended for CAHPS average star rating of 4, or an optimal contract sample size of 3350 is recommended for CAHPS average star rating of 4.5.

FIG. 4B is a graph that illustrates the impact of sample size on cutpoint star volatility, according to aspects of the disclosure. In one instance, the CAHPS surveys deal with a sample of the population, hence despite computing the true population mean, there may be some sampling variability for the scores. During data analysis, cutpoint is a specified value to sort continuous variables into discrete categories. As the sample size increases, the mean sampling distribution tightens, and this impacts the probability of the sample mean interacting with measure cutpoints. In one embodiment, the optimization platform 111, utilizing the user interface module 209, generates a graph 403 in the UE 103 associated with the user 102 to demonstrate the impact of sample size on cut-point star volatility. In this example embodiment, graph 403 depicts a sampling distribution of a contract with a sample size of 800, wherein the probability density function (PDF) of the sampling distribution is measured against the response rate.

FIG. 5 is a graph that illustrates sampling distribution for a contract-measure for determining an expected star value, according to aspects of the disclosure. In one embodiment, the optimization platform 111 executes the following steps for each allowable sample size within a contract-measure:
1. Given some inputs (e.g., the mean and standard deviation for the measure, which can be predictions or some output of a proxy survey) the optimization platform 111 establishes an appropriate sampling distribution for a given sample size (which is approximated as normal according to the central limit theorem);
2. The optimization platform 111 recalculates the significance and reliability thresholds from statistical tests;
3. Using the cumulative distribution function (CDF) of the sampling distribution, the optimization platform 111 calculates the probability of obtaining a given star value based on the parameters of the CMS adjustment grid; and
4. The optimization platform 111 calculates the expected star value for the measure by taking the sum of the product of the probability and the star value itself.

In one instance, for each allowable sample size, the optimization platform 111 illustrates the graph of the PDF of the sampling distribution S in chart 501. The optimization platform 111 calculates the probability of getting a particular star by partitioning the distribution into measured cutpoints (e.g., c2, c3, c4, and c5), sig_{above/below} thresholds, and new reliability assignment according to the adjustment grid 325. The optimization platform 111 calculates the area under the curve (e.g., areas 1, 2, 3, 4, and 5) to obtain the probability of the contract attaining each star rating. In one embodiment, a sum of the probability by the star value (based on the above-referenced equation 5) is selected. In this instance, the sum of the probability by the star value is 88 which gives a base star value of 4.

FIG. 6A is a user interface diagram that illustrates an optimal sample size, according to aspects of the disclosure. In one example embodiment, the optimization platform 111 performs eight different measures (e.g., coordination of care (COC) 601, getting care quickly (GCQ) 603, getting needed care (GNC) 605, getting needed prescription drugs (GNPD) 607, health plan customer service (HPCS) 609, rating drug plan (RDP) 611, rating health care (RHC) 613, and rating health plan (RHP) 615) for a contract to determine measure level expected stars by sample size. The optimization platform 111, via the user interface module 209, generates a presentation of the eight different measures in a user interface of the UE 103. As illustrated, out of the eight measures, GNC 605 and RDP 611 demonstrate an increase in sample size results in higher star value, whereas GCQ 603, GNPD 607, and HPCS 609 show an increase in sample size results in the lowest star value. The optimization platform 111 finds a balance between these different measures because the contract may perform better on some measures but worse on others. The optimization platform 111 aggregates the results of each of the measures to calculate a contract level expected star 617.

In one embodiment, the optimization platform 111, via the user interface module 209, generates a presentation of the contract level expected star 617 in a user interface 618 of the UE 103. In one instance, user interface 618 includes several interactive areas and/or clickable icons that, upon selection direct the user 102 to a separate screen including the eight different measures. In another instance, user interface 618 color codes the contract level expected star 617 according to the preset configurations, and highlights the optimal sample size in the contract level expected star 617 for the contract. As illustrated, the optimization platform 111 outputs a myriad of values, and the user 102 can ultimately decide which sample size to use for a contract within the CMS star ratings program. For example, user interface 618 shows that the optimal sample size is in the region of 1700, but the user 102 may choose any of the eight different measures.

FIG. 6B is a user interface diagram that illustrates an expected contract star by sample size and mean adjustment using a parametric method, according to aspects of the disclosure. In one embodiment, the optimization platform 111, via the user interface module 209, generates a presentation of expected contract stars for eleven different measures (e.g., measures 619 through 639). As depicted, measures 619, 621, 623, 625, and 627 have negative contract mean adjustments. Measure 629 is a base case without any mean adjustment. Measures 631 through 639 have positive contract mean adjustments. In one instance, the optimization platform 111 may execute one or more algorithms to shift (e.g., up or down) the mean input for each of the measures 619, 621, 623, 625, and 627 by a constant value. For example, in measure 619 the mean is shifted by negative 1 unit, whereas in measure 639 the mean is shifted by positive 1 unit. For example, in measure 621 the mean is shifted by negative 0.8 unit, whereas in measure 637 the mean is shifted by positive 0.8 unit.

In general, any process or operation discussed in this disclosure is understood to be computer-implementable, such as the process illustrated in FIG. 3A are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

FIG. 7 illustrates an implementation of a computer system that executes techniques presented herein. A computer system 700 includes a set of instructions that are executed to cause the computer system 700 to perform any one or more of the methods or computer based functions disclosed herein. The computer system 700 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 700 operates in the capacity of a server or as a client user computer in a server-client user network environment, or as a peer computer system in a peer-to-peer (or distributed) network environment. The computer system 700 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 700 is implemented using electronic devices that provide voice, video, or data communication. Further, while the computer system 700 is illustrated as a single system, the term “system” shall also be taken to include any collection of systems or sub-systems that individually or jointly execute a set, or multiple sets, of instructions to perform one or more computer functions.

As illustrated in FIG. 7, the computer system 700 includes a processor 702, e.g., a central processing unit (CPU), a graphics processing unit (GPU), or both. The processor 702 is a component in a variety of systems. For example, the processor 702 is part of a standard personal computer or a workstation. The processor 702 is one or more processors, digital signal processors, application specific integrated circuits, field programmable gate arrays, servers, networks, digital circuits, analog circuits, combinations thereof, or other now known or later developed devices for analyzing and processing data. The processor 702 implements a software program, such as code generated manually (e.g., programmed).

The computer system 700 includes a memory 704 that communicates via a bus 708. The memory 704 is a main memory, a static memory, or a dynamic memory. The memory 704 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 704 includes a cache or random-access memory for the processor 702. In alternative implementations, the memory 704 is separate from the processor 702, such as a cache memory of a processor, the system memory, or other memory. Memory 704 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 704 is operable to store instructions executable by the processor 702. The functions, acts, or tasks illustrated in the figures or described herein are performed by processor 702 executing the instructions stored in the memory 704. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 700 further includes a display 710, such as a liquid crystal display (LCD), an organic light emitting diode (OLED), a flat panel display, a solid-state display, a cathode ray tube (CRT), a projector, a printer or other now known or later developed display device for outputting determined information. The display 710 acts as an interface for the user to see the functioning of the processor 702, or specifically as an interface with the software stored in the memory 704 or in a drive unit 706.

Additionally or alternatively, the computer system 700 includes an input/output device 712 configured to allow a user to interact with any of the components of the computer system 700. The input/output device 712 is a number pad, a keyboard, a cursor control device, such as a mouse, a joystick, touch screen display, remote control, or any other device operative to interact with the computer system 700.

The computer system 700 also includes the drive unit 706 implemented as a disk or optical drive. The drive unit 706 includes a computer-readable medium 722 in which one or more set of instructions 724, e.g. software, is embedded. Further, the set of instructions 724 embodies one or more of the methods or logic as described herein. The set of instructions 724 resides completely or partially within the memory 704 and/or within the processor 702 during execution by the computer system 700. The memory 704 and the processor 702 also include computer-readable media as discussed above.

In some systems, the computer-readable medium 722 includes the set of instructions 724 or receives and executes the set of instructions 724 responsive to a propagated signal so that a device connected to network 730 communicates voice, video, audio, images, or any other data over network 730. Further, the set of instructions 724 are transmitted or received over the network 730 via a communication port or interface 720, and/or using the bus 708. The communication port or interface 720 is a part of the processor 702 or is a separate component. The communication port or interface 720 is created in software or is a physical connection in hardware. The communication port or interface 720 is configured to connect with the network 730, external media, the display 710, or any other components in the computer system 700, or combinations thereof. The connection with network 730 is a physical connection, such as a wired Ethernet connection, or is established wirelessly as discussed below. Likewise, the additional connections with other components of the computer system 700 are physical connections or are established wirelessly. Network 730 alternatively be directly connected to the bus 708.

While the computer-readable medium 722 is shown to be a single medium, the term “computer-readable medium” includes a single medium or multiple media, such as a centralized or distributed database, and/or associated caches and servers that store one or more set of instructions. The term “computer-readable medium” also includes any medium that is capable of storing, encoding, or carrying a set of instructions for execution by a processor or that causes a computer system to perform any one or more of the methods or operations disclosed herein. The computer-readable medium 722 is non-transitory, and may be tangible.

The computer-readable medium 722 includes a solid-state memory such as a memory card or other package that houses one or more non-volatile read-only memories. The computer-readable medium 722 is a random-access memory or other volatile re-writable memory. Additionally or alternatively, the computer-readable medium 722 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

The computer system 700 is connected to network 730. Network 730 defines one or more networks including wired or wireless networks. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. Network 730 includes wide area networks (WAN), such as the Internet, local area networks (LAN), campus area networks, metropolitan area networks, a direct connection such as through a Universal Serial Bus (USB) port, or any other networks that allows for data communication. Network 730 is configured to couple one computing device to another computing device to enable communication of data between the devices. Network 730 is generally enabled to employ any form of machine-readable media for communicating information from one device to another. Network 730 includes communication methods by which information travels between computing devices. Network 730 is divided into sub-networks. The sub-networks allow access to all of the other components connected thereto or the sub-networks restrict access between the components. Network 730 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (e.g., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.

Example 1. A system for an automated process for determining an optimal sample size for a contract, comprising: one or more processors; and at least one non-transitory computer readable medium storing instructions which, when executed by the one or more processors, cause the one or more processors to perform operations comprising: receiving response data from data collection objects, wherein the response data includes response scores to queries in the data collection objects; determining a sampling distribution for each of one or more sample sizes based on the response scores; determining a significance threshold and a reliability coefficient for each of the one or more sample sizes; determining a probability for each of one or more star values for the one or more sample sizes based on a cumulative distributive function (CDF) of the sampling distribution and one or more parameters of an adjustment grid; determining an expected star value for each of the one or more sample sizes based on the probability determined for each of the one or more star values; and causing a presentation of the expected star value determined for each of the one or more sample sizes in a user interface of a device, wherein at least one of the one or more sample sizes with a highest expected star value is recommended as the optimal sample size.

Example 2. The system of example 1, wherein determining the probability for each star value comprises: determining a curve indicating the sampling distribution for the one or more sample sizes; and determining a probability of the contract achieving each star value by determining an area under the curve of a probability density function of the sampling distribution, wherein the curve is partitioned by at least one of a cutpoints, a significance threshold, or a reliability assignment according to the adjustment grid.

Example 3. The system of any of the preceding examples, wherein determining the expected star value comprises: determining a mean across each expected measure star, wherein the expected measure star is a product of the probability for each star value and the star value.

Example 4. The system of any of the preceding examples, wherein the user interface includes a visual representation of the expected star value determined for each of the one or more sample sizes, as a function of the one or more sample sizes at a contract level and a measure level.

Example 5. The system of any of the preceding examples, wherein the significance threshold is an output of a two sided t-test that compares a mean of the contract to an average of one or more other contracts.

Example 6. The system of any of the preceding examples, wherein the reliability coefficient compares a variance of the contract to variances between one or more other contracts.

Example 7. The system of example 2, further comprising: determining a standard error for each of the one or more sample sizes, wherein the standard error measures variation in contract values, and wherein the standard error is based on the response scores and a user size.

Example 8. The system of example 7, wherein each contract value is a mean of the response scores to the queries in the data collection objects.

Example 9. The system of example 7, wherein the user size is a product of a response rate and a sample size.

Example 10. The system of any of the preceding examples, wherein the data collection objects include a Consumer Assessment of Health Care Providers and Systems (CAHPS).

Example 11. A computer-implemented method for an automated process for determining an optimal sample size for a contract, the method comprising: receiving, by one or more processors, response data from data collection objects, wherein the response data includes response scores to queries in the data collection objects; determining, by the one or more processors, a sampling distribution for each of one or more sample sizes based on the response scores; determining, by the one or more processors, a significance threshold and a reliability coefficient for each of the one or more sample sizes; determining, by the one or more processors, a probability for each of one or more star values for the one or more sample sizes based on a cumulative distributive function (CDF) of the sampling distribution and one or more parameters of an adjustment grid; determining, by the one or more processors, an expected star value for each of the one or more sample sizes based on the probability determined for each of the one or more star values; and causing, by the one or more processors, a presentation of the expected star value calculated for each of the one or more sample sizes in a user interface of a device, wherein at least one of the one or more sample sizes with a highest expected star value is recommended as the optimal sample size.

Example 12. The computer-implemented method of example 11, wherein determining the probability for each star value comprises: determining, by the one or more processors, a curve indicating the sampling distribution for the one or more sample sizes; and determining, by the one or more processors, a probability of the contract achieving each star value by determining an area under the curve of a probability density function of the sampling distribution, wherein the curve is partitioned by at least one of a cutpoints, a significance threshold, or a reliability assignment according to the adjustment grid.

Example 13. The computer-implemented method of any of examples 11-12, wherein determining the expected star value comprises: determining, by the one or more processors, a mean across each expected measure star, wherein the expected measure star is a product of the probability for each star value and the star value.

Example 14. The computer-implemented method of any of examples 11-13, wherein the user interface includes a visual representation of the expected star value determined for each of the one or more sample sizes, as a function of the one or more sample sizes at a contract level and a measure level.

Example 15. The computer-implemented method of any of examples 11-14, wherein the significance threshold is an output of a two sided t-test that compares a mean of the contract to an average of one or more other contracts.

Example 16. The computer-implemented method of any of examples 11-15, wherein the reliability coefficient compares a variance of the contract to variances between one or more other contracts.

Example 17. The computer-implemented method of example 12, further comprising: determining, by the one or more processors, a standard error for each of the one or more sample sizes, wherein the standard error measures variation in contract values, and wherein the standard error is based on the response scores and a user size.

Example 18. A non-transitory computer readable medium for an automated process for determining an optimal sample size for a contract, the non-transitory computer readable medium storing instructions which, when executed by one or more processors, cause the one or more processors to perform operations comprising: receiving response data from data collection objects, wherein the response data includes response scores to queries in the data collection objects; determining a sampling distribution for each of one or more sample sizes based on the response scores; determining a significance threshold and a reliability coefficient for each of the one or more sample sizes; determining a probability for each of one or more star values for the one or more sample sizes based on a cumulative distributive function (CDF) of the sampling distribution and one or more parameters of an adjustment grid;

determining an expected star value for each of the one or more sample sizes based on the probability determined for each of the one or more star values; and causing a presentation of the expected star value determined for each of the one or more sample sizes in a user interface of a device, wherein at least one of the one or more sample sizes with a highest expected star value is recommended as the optimal sample size.

Example 19. The non-transitory computer readable medium of example 18, wherein determining the probability for each star value comprises: determining a curve indicating the sampling distribution for the one or more sample sizes; and determining a probability of the contract achieving each star value by determining an area under the curve of a probability density function of the sampling distribution, wherein the curve is partitioned by at least one of a cutpoints, a significance threshold, or a reliability assignment according to the adjustment grid.

Example 20. The non-transitory computer readable medium of any of examples 18-19, wherein determining the expected star value comprises: determining a mean across each expected measure star, wherein the expected measure star is a product of the probability for each star value and the star value.

What is claimed is:

1. A system for an automated process for determining an optimal sample size for a contract, comprising:
one or more processors; and
at least one non-transitory computer readable medium storing instructions which, when executed by the one or more processors, cause the one or more processors to perform operations comprising:
receiving response data from data collection objects, wherein the response data includes response scores to queries in the data collection objects;
determining a sampling distribution for each of one or more sample sizes based on the response scores;
determining a significance threshold and a reliability coefficient for each of the one or more sample sizes;
determining a probability for each of one or more star values for the one or more sample sizes based on a cumulative distributive function (CDF) of the sampling distribution and one or more parameters of an adjustment grid;
determining an expected star value for each of the one or more sample sizes based on the probability determined for each of the one or more star values; and
causing a presentation of the expected star value determined for each of the one or more sample sizes in a user interface of a device, wherein at least one of the one or more sample sizes with a highest expected star value is recommended as the optimal sample size.

2. The system of claim 1, wherein determining the probability for each star value comprises:
determining a curve indicating the sampling distribution for the one or more sample sizes; and
determining a probability of the contract achieving each star value by determining an area under the curve of a probability density function of the sampling distribution, wherein the curve is partitioned by at least one of a cutpoints, a significance threshold, or a reliability assignment according to the adjustment grid.

3. The system of claim 1, wherein determining the expected star value comprises:
determining a mean across each expected measure star, wherein the expected measure star is a product of the probability for each star value and the star value.

4. The system of claim 1, wherein the user interface includes a visual representation of the expected star value determined for each of the one or more sample sizes, as a function of the one or more sample sizes at a contract level and a measure level.

5. The system of claim 1, wherein the significance threshold is an output of a two sided t-test that compares a mean of the contract to an average of one or more other contracts.

6. The system of claim 1, wherein the reliability coefficient compares a variance of the contract to variances between one or more other contracts.

7. The system of claim 2, further comprising:
determining a standard error for each of the one or more sample sizes,
wherein the standard error measures variation in contract values, and wherein the standard error is based on the response scores and a user size.

8. The system of claim 7, wherein each contract value is a mean of the response scores to the queries in the data collection objects.

9. The system of claim 7, wherein the user size is a product of a response rate and a sample size.

10. The system of claim 1, wherein the data collection objects include a Consumer Assessment of Health Care Providers and Systems (CAHPS).

11. A computer-implemented method for an automated process for determining an optimal sample size for a contract, the method comprising:
receiving, by one or more processors, response data from data collection objects, wherein the response data includes response scores to queries in the data collection objects;
determining, by the one or more processors, a sampling distribution for each of one or more sample sizes based on the response scores;
determining, by the one or more processors, a significance threshold and a reliability coefficient for each of the one or more sample sizes;
determining, by the one or more processors, a probability for each of one or more star values for the one or more sample sizes based on a cumulative distributive function (CDF) of the sampling distribution and one or more parameters of an adjustment grid;
determining, by the one or more processors, an expected star value for each of the one or more sample sizes based on the probability determined for each of the one or more star values; and
causing, by the one or more processors, a presentation of the expected star value calculated for each of the one or more sample sizes in a user interface of a device, wherein at least one of the one or more sample sizes with a highest expected star value is recommended as the optimal sample size.

12. The computer-implemented method of claim 11, wherein determining the probability for each star value comprises:
determining, by the one or more processors, a curve indicating the sampling distribution for the one or more sample sizes; and
determining, by the one or more processors, a probability of the contract achieving each star value by determining an area under the curve of a probability density function of the sampling distribution, wherein the curve is partitioned by at least one of a cutpoints, a significance threshold, or a reliability assignment according to the adjustment grid.

13. The computer-implemented method of claim 11, wherein determining the expected star value comprises:
determining, by the one or more processors, a mean across each expected measure star, wherein the expected measure star is a product of the probability for each star value and the star value.

14. The computer-implemented method of claim 11, wherein the user interface includes a visual representation of the expected star value determined for each of the one or more sample sizes, as a function of the one or more sample sizes at a contract level and a measure level.

15. The computer-implemented method of claim 11, wherein the significance threshold is an output of a two sided t-test that compares a mean of the contract to an average of one or more other contracts.

16. The computer-implemented method of claim 11, wherein the reliability coefficient compares a variance of the contract to variances between one or more other contracts.

17. The computer-implemented method of claim 12, further comprising:
determining, by the one or more processors, a standard error for each of the one or more sample sizes,
wherein the standard error measures variation in contract values, and wherein the standard error is based on the response scores and a user size.

18. A non-transitory computer readable medium for an automated process for determining an optimal sample size for a contract, the non-transitory computer readable medium storing instructions which, when executed by one or more processors, cause the one or more processors to perform operations comprising:
receiving response data from data collection objects, wherein the response data includes response scores to queries in the data collection objects;
determining a sampling distribution for each of one or more sample sizes based on the response scores;
determining a significance threshold and a reliability coefficient for each of the one or more sample sizes;
determining a probability for each of one or more star values for the one or more sample sizes based on a cumulative distributive function (CDF) of the sampling distribution and one or more parameters of an adjustment grid;
determining an expected star value for each of the one or more sample sizes based on the probability determined for each of the one or more star values; and
causing a presentation of the expected star value determined for each of the one or more sample sizes in a user interface of a device, wherein at least one of the one or more sample sizes with a highest expected star value is recommended as the optimal sample size.

19. The non-transitory computer readable medium of claim 18, wherein determining the probability for each star value comprises:
determining a curve indicating the sampling distribution for the one or more sample sizes; and
determining a probability of the contract achieving each star value by determining an area under the curve of a probability density function of the sampling distribution, wherein the curve is partitioned by at least one of a cutpoints, a significance threshold, or a reliability assignment according to the adjustment grid.

20. The non-transitory computer readable medium of claim 18, wherein determining the expected star value comprises:
determining a mean across each expected measure star, wherein the expected measure star is a product of the probability for each star value and the star value.
